(19) United States
(12) Patent Application Publication
(10) Pub. No.: US 2005/0183722 A1
(43) Pub. Date: Aug. 25, 2005

(54) External chest therapy blanket for infants

(76) Inventor: Jagadish Bilgi, Ankeny, IA (US)

Correspondence Address: STURM & FIX LLP, 206 SIXTH AVENUE, SUITE 1213, DES MOINES, IA, 50309-4076, US

(21) Appl. No.: 11/111,383

(22) Filed: Apr. 21, 2005

Related U.S. Application Data
(63) Continuation-in-part of application No. 10/259,895, filed on Sep. 27, 2002.

(51) Int. Cl. A61M 15/00; A61M 16/10

(52) U.S. Cl. 128/203.12

(57) ABSTRACT

Massaging and compressing the lungs of a patient has been proven effective in loosening mucus buildup. Devices and methods for this purpose have been designed for patients older than infants and toddlers. A method for massaging and compressing the lungs of infants and toddlers has been developed using a device similar to those used for older patients. Such devices use pulsating air to inflate and deflate bladders that are in contact with the patient and apply pressure only to the upper torso of the patient. For older patients, the devices are typically made into a vest and fitted on the patient. For infants and toddlers the device, herein called a J-blanket™, having the same bladder arrangement, is spread flat on a suitable surface and the patient placed on the J-blanket. This reduces the physical trauma to these very young patients. The J-blanket can be fitted with sensors to provide important feedback related to the patient's well-being. The same method can be applied to patients of any age that might experience too great a physical trauma when treated in the conventional fashion.

### CROSS REFERENCE TO RELATED APPLICATIONS

This application is a Continuation-in-Part of co-pending U.S. patent application Ser. No. 10/259,895, filed Sep. 27, 2002 and entitled EXTERNAL CHEST THERAPY BLANKET FOR INFANTS, which is incorporated by reference herein in its entirety.

### BACKGROUND OF THE INVENTION

1. Field of the Invention

The present invention relates generally to a medical a device and the use thereof. More particularly the present invention relates to a device for compressing and massaging the upper torso of patients for the purpose of loosening mucus from the lungs of the patient. It has application to patients that are young (infants to toddlers), or would experience undo physical trauma undergoing more traditional treatment. Furthermore, this invention has particular application to infants afflicted with cystic fibrosis, pneumonia, asthma, and immature lungs.

2. Background Art

A buildup of mucus in the lungs of a patient, whose immune system may be compromised, anyway, can be dangerous and even fatal. Removal of the mucus helps in the functioning of the lungs, and in reducing the chance of infection within the lungs.

A device used presently for the purpose of massaging the lungs is revealed in Warwick et al. (U.S. Pat. No. 5,056,505), which is incorporated herein by reference. These present-day devices utilize air to inflate a bladder or bladders connected in the form of a vest or sleeveless garment worn on the upper body of the patient. Such a garment has been found to be too bulky for young infant or toddler patients. Young patients, especially prematurely born infants, require specialized equipment for this purpose.

Another device for massaging the lungs of a patient is disclosed in U.S. Pat. No. 6,098,222 by Hand et al. This device comprises a plurality of sacks arranged transversely to the patient, the plurality of sacks extending the length of the patient's body. The sacks are disposed on a rigid support frame, therefore, the system lacks portability. Pulsations and vibrations applied to the head and/or kidney regions of an infant or toddler patient are likely to result in hemorrhaging. Therefore, an adult device that is simply resized for use with smaller patients is unsuitable and even dangerous for infants and toddlers.

Present-day devices for massaging and compressing the lungs use air, carried to and from the device via air ducts. The air compression system has been found to be too vigorous for very young patients. Hence, the teaching in the known prior today does not lead one to an acceptable solution for very young patients from premature babies on through toddlers.

For the above reasons, there is a need for a method for massaging the upper torso of a toddler, infant, or even a premature baby. There is also a need for the device used to be driven by an air-pumping system that is (or can be) regulated for infant and toddler human beings.

### SUMMARY OF THE INVENTION

A purpose of this invention is to provide a method to reduce physical trauma, for compressing and massaging a small patient's upper torso to loosen a buildup of mucus in the patient's lungs. These patients, in particular are infants or toddlers, but can include anyone for whom traditional lung compression methods provide too much physical trauma.

A further purpose, in conjunction with the above, is to provide an apparatus for modulating pressure pulses to a bladder used in the device for compressing and massaging the patient's upper torso.

Another purpose, related to the previous purpose, is to provide an apparatus that limits the manipulations to the upper torso of the patient.

Still another purpose of this invention is to provide, in conjunction with the above device for compressing and massaging a patient's upper torso, built-in sensors for the small patient's oxygen level, heart rate, and respiration rate.

There are many afflictions for which treatment might include procedures for loosening mucus from the lungs. Such afflictions include premature lungs, pneumonia, cystic fibrosis, and asthma.

For small patients, a preferred approach is one utilizing a J-blanket™ that the patient lies on, either face up, face down, or on the side. In these cases, the J-blanket need not be wrapped around the patient at all, significantly improving the child's comfort. The J-blanket may also be placed over the patient. The J-blanket is portable, in that it may be carried by a single health-care worker and spread on any surface or patient.

The J-blanket is outfitted with multiple chambers, all of which are expanded when filled with air at a higher pressure than atmospheric. Pressure relief valves are also incorporated at various locations on the J-blanket. Alternating expansion and contraction of the chambers produces the desired effect of compressing and massaging the patient's lungs.

An air handling system for the J-blanket is required to carry compressed air to and from the J-blanket. The special needs of very small patients—infants and toddlers—require that the air handling system provides a level of compression and massaging that they can tolerate.

Especially for very young patients, there is a need to monitor critical patient parameters. Sensors can be mounted on or in the J-blanket to monitor the patient's oxygen saturation, heart rate and respiration rate. Because the J-blanket must be in close contact with the patient to serve the purpose of massaging the upper torso, the sensors are, by default, in close contact with the patient. Massaging need not be taking place for the sensors to be active. Therefore, there is advantage to keeping the patient on the J-blanket even when there is not a need to loosen mucus from the lungs.

### BRIEF DESCRIPTION OF THE DRAWINGS

FIG. 1 shows a J-blanket, under a patient, with a system for providing compressed air and readouts for sensors.

FIG. 2 shows a more detailed view of the system for providing compressed air and readouts for sensors.

FIG. 3 shows the side of a J-blanket that faces toward from the patient.

FIG. 4 shows the side of a J-blanket that faces away from the patient.

FIG. 5 shows the annular tubes for handling air to and from the J-blanket.

FIG. 6 shows pressure relief valve.

FIG. 7 shows a sensor mounted in a J-blanket surface.

FIG. 8 shows construction of the material of which J-blanket is made.

FIG. 9 shows a J-blanket wrapped around a patient.

### BEST MODE FOR CARRYING OUT THE INVENTION

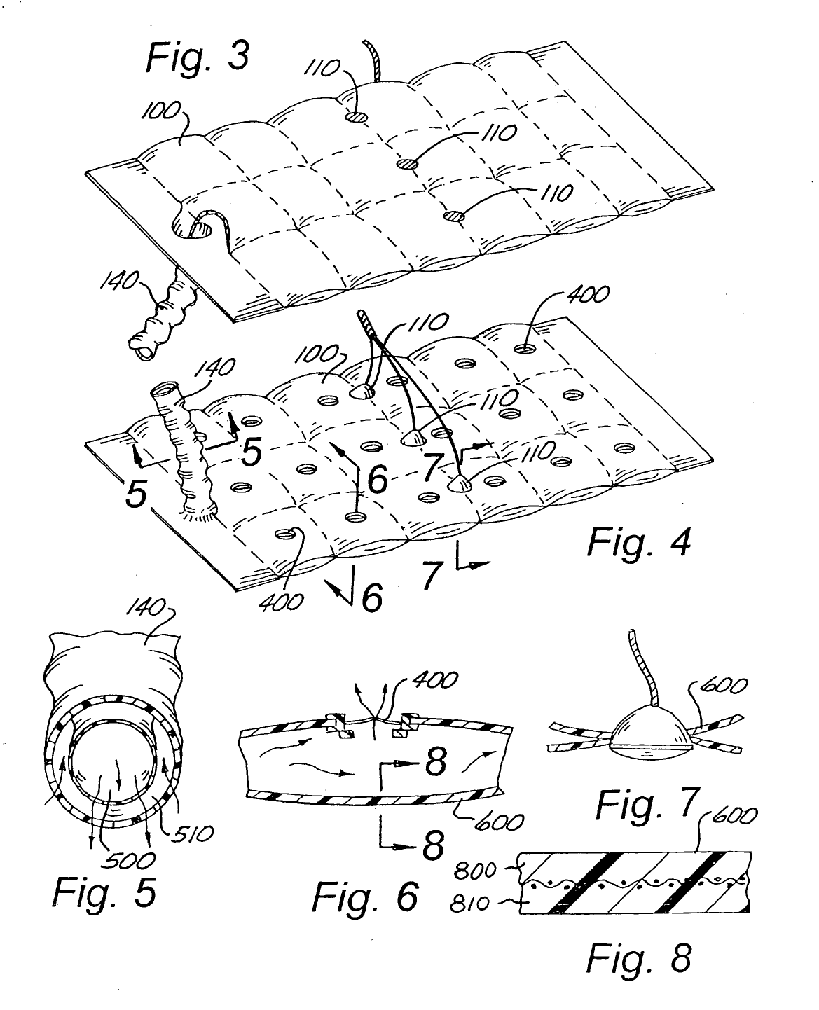

Experience has shown that providing alternating pressure to the chest and/or upper torso area of a patient with a buildup of mucus is effective in reducing that mucus buildup. Accordingly, devices have been in use to effect the massage and compression of the lungs of patients in need of such treatment. To treat small human patients (from premature babies to toddlers), or any patient for whom traditional lung massage would be too physically traumatic, a device, illustrated in FIGS. 3 and 4 and called a J-blanket for massaging and compressing the lungs can be spread on a surface such as a table or bed and the child placed on the J-blanket. Alternatively, the J-blanket may be spread on the patient. The operative portion of the J-blanket, that is, all the portion of the J-blanket that can be operative, is sized such that only the upper torso of the patient is disposed on the J-blanket.

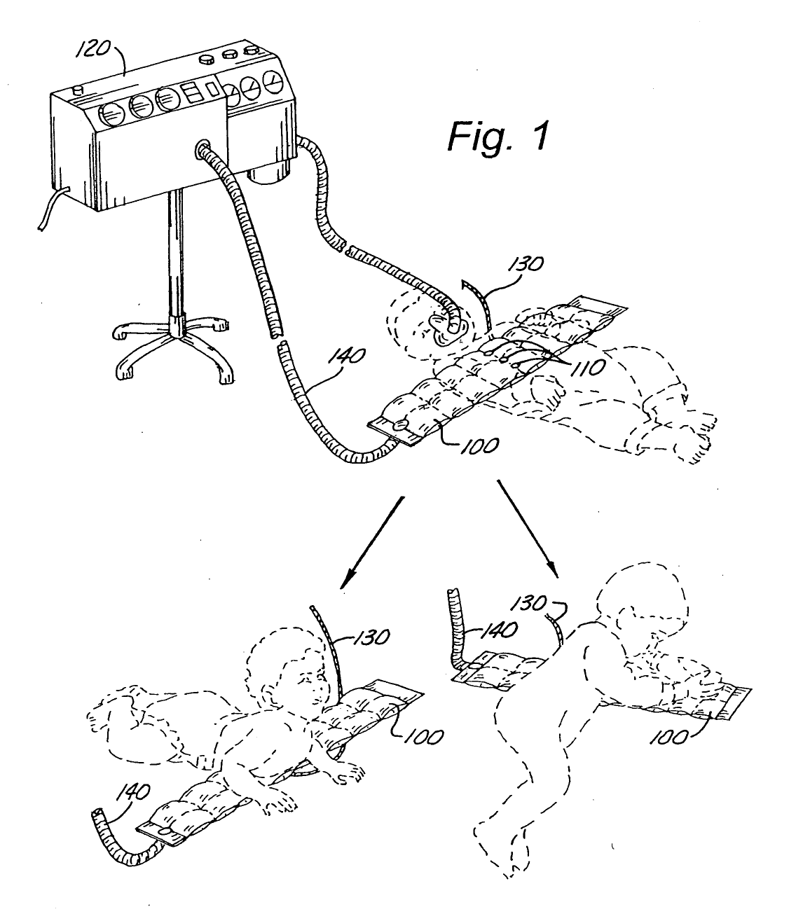

An overview of the lung massaging system is illustrated in FIG. 1. The J-blanket 100 is shown lying on a surface. There are three sensors 110 to monitor the patient's oxygen level, heart rate, and respiration rate. These data are transmitted to the air-handling and monitoring unit 120 via wires 130. Pulsating air comes from and returns to the air handling unit via an annular hose 140. An annular hose 140 is defined herein as a hose having two passageways for fluid to pass. A first passageway 500 (see FIG. 5) is within the inner hose, shown with a round cross-section in FIG. 5. The second passageway is the annulus 510 between the inner and outer hose walls. This definition will be used throughout this document, including the claims.

As revealed in FIG. 1, a very small, young, human patient may be treated most successfully, with the least physical trauma, by placing the infant or toddler on the J-blanket 100 rather than putting a vest on the patient.

Note that the J-blanket 100 manipulates only the upper torso of the patient. Vibrating or pulsating a very young patient's head or kidneys may cause hemorrhaging of those body parts and subsequent damage or even death. Therefore, a device providing manipulation of more than the upper torso of the patient is not prudent for very young patients.

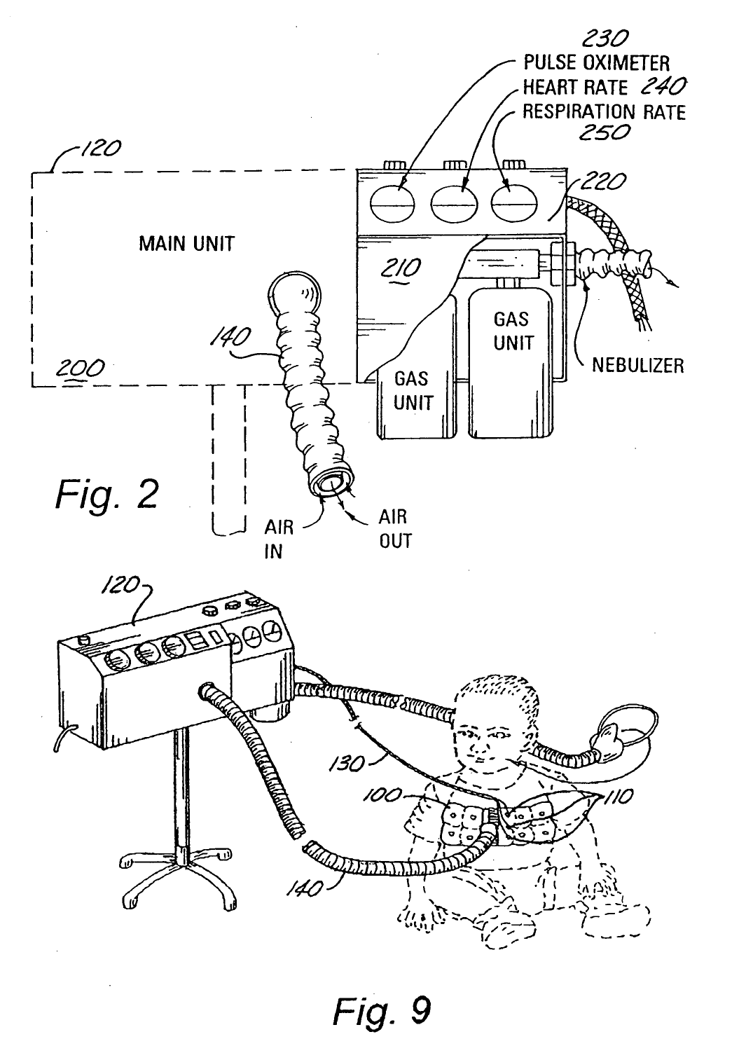

A more detailed view of the air handling unit 120 is shown in FIG. 2. The air handling unit 120 comprises three separate parts:
1. A main unit 200 providing the compressed and pulsating air for the J-blanket 100 via the annular hose 140.
2. A respirator unit 210 providing moisturized (and medicated, if necessary) air for the patient's respiration.
3. A monitoring unit 220 providing readouts for the sensors 110 mounted on or in the J-blanket 100, as well as appropriate alarms based on the readings of the sensors 110.

The monitoring unit 220 has a monitor 230 for oxygen saturation, a monitor 240 for pulse rate, and a monitor 250 for respiration rate. Generally, the monitors 220, 230, 240 include readouts as well as alarms.

FIG. 3 shows a view of a J-blanket 100 showing the side facing the patient.

FIG. 4 shows a view of a J-blanket 100 showing the side facing away from the patient. The sensors 110 and the annular hose 140 are shown. The sensors 110 are placed in the center of J-blanket 100 so they are in contact with the patient.

The annular hose 140 is detailed in FIG. 5. Air passing from main unit 200 to J-blanket 100 travels in the circular center region 500 while air passing from J-blanket 100 to main unit 200 travels in the annular region 510. These directions can be reversed and this invention is not limited to either possibility.

Pressure relief valves 400 in J-blanket 100 are shown in FIG. 4 and detailed in FIG. 6. The pressure relief valve 400 provides protection to the J-blanket 100 in the event of overpressuring.

One of the sensors 110 is shown in more detail in FIG. 7. It is mounted in the material 600 making up the J-blanket 100, but it could be mounted on the material 600, or even under it.

FIG. 8 shows details of the material 600 making up the J-blanket 100. It is comprised of two layers of material. The first material 800 is for the comfort of the patient, so is soft but may not have the strength to sustain the requisite pulsating loads. The second material 810 is for the strength required to withstand the stresses applied by the pressurized air.

In FIG. 9, the J-blanket 100 is shown being used with the J-blanket 100 wrapped around the patient's chest. For this usage, hook and loop fasteners like the Velcro® brand of fastener are used at the ends so the blanket 100 can be kept on the patient's chest. Other aspects of the blanket are the same as above. The sensors 110 are connected to the monitor unit 220 via wires 130. The pulsating air is passed to and from the blanket 100 through the annular hose 140.

Obviously many modifications and variations of the present invention are possible in light of the above teachings. It is, therefore, to be understood that within the scope of the appended claims, the invention may be practiced otherwise than as specifically described.

1. A method for using a blanket for massaging and compressing lungs of a human patient for the purpose of loosening mucus, the method comprising:
(a) spreading the blanket for massaging and compressing the lungs of the patient out on a surface;
(b) laying the patient on the blanket;
(c) limiting the blanket to an upper torso of the patient; and
(d) providing pulsations for massaging and compressing the lungs of the patient.

2. The method of claim 1 wherein the patient lies face up, said blanket limited to an upper back of the patient.

3. The method of claim 1 wherein the patient lies face down, said blanket limited to a chest of the patient.

4. The method of claim 1 wherein the patient lies on their side, said blanket limited to the upper torso of the side of the patient.

5. The method of claim 1 wherein the blanket for massaging and compressing the lungs of the human patient is used with sensors for critical health parameters.

6. The method of claim 5 wherein the sensors used include a sensor for measuring oxygen saturation in blood.

7. The method of claim 5 wherein the sensors used include a sensor for measuring pulse rate.

8. The method of claim 5 wherein the sensors used include a sensor for measuring respiration rate.

9. The method of claim 1 wherein pulsating air flow passes through an annular hose to the blanket for massaging and compressing lungs of a human patient, said annular hose including an outer annular wall and an inner annular wall inside the outer annular wall thereby forming a first passageway between the first and second annular wall and a second passageway inside the second annular wall.

10. The method of claim 9 wherein the pulsating air flow is provided by pulsating air pressure supplied by a main air unit to the blanket for massaging and compressing lungs of a human patient, a strength of the pressure pulsations being tempered for infants and toddlers.

11. The method of claim 1 wherein providing pulsations for massaging and compressing the lungs of the patient comprises causing the blanket to pulsate due to air pressure changes inside of a sealed bladder.

12. An apparatus for massaging and compressing the lungs of a human infant comprising:
a blanket disposed substantially completely on a substantially flat surface, said blanket comprising at least one sealed chamber and adapted to have the human infant lie thereon with the infant's upper torso being in juxtaposition to the at least one sealed chamber of the blanket, said blanket being further sized such that an operative portion of the blanket, being defined as an entire portion of the blanket that can be made to be operative, contacts only an upper torso of the patient; and
compressed air means for selectively inflating and deflating the at least one sealed chamber of the blanket.

13. The apparatus of claim 12 wherein the blanket for massaging and compressing lungs of a human patient includes sensors for critical health parameters.

14. The method of claim 13 wherein the sensors include a sensor for measuring oxygen saturation in blood.

15. The method of claim 13 wherein the sensors include a sensor for measuring pulse rate.

16. The method of claim 13 wherein the sensors include a sensor for measuring respiration rate.

17. A method for massaging and compressing lungs of a patient for the purpose of loosening mucus using a pneumatically actuated blanket comprising a plurality of air bladders attached one to another, said blanket being portable with respect to a surface on which the blanket is spread, the method comprising:
(a) spreading the portable, pneumatically actuated blanket of attached air bladders onto the surface;
(b) disposing the patient on the blanket such that any of the plurality of inflatable bladders in contact with the patient contact only an upper torso of the patient; and
(c) actuating said portable, pneumatically actuated blanket in a pulsating manner to massage and compress the lungs of a patient.

18. The method of claim 1 wherein the blanket is portable with respect to the surface on which it is spread like a blanket and the step of spreading the blanket out on a surface comprises spreading the portable blanket out on a surface.

19. The method of claim 1 wherein the blanket comprises a plurality of inflatable bladders attached one to another.

20. A method of using an apparatus for massaging and compressing the lungs of a human patient, said apparatus comprising a blanket disposed substantially completely on a substantially flat surface, said blanket having an operative portion thereof comprising an entire portion that can be made to be operative and which comprises at least one sealed chamber therein and adapted to have an infant lie thereon with the infant's chest being in juxtaposition to the at least one sealed chamber of the blanket; and a source of compressed air operatively connected to the sealed chamber for selectively inflating and deflating the blanket; the operative portion of the blanket being a width no greater than a distance between shoulders and waist of the human patient; said method comprising:
(a) spreading the blanket on the flat surface for massaging and compressing the lungs of a patient out on a surface;
(b) laying a torso portion of a patient on the blanket; and
(c) using the source of compressed air to pulsate the sealed chamber to cause vibration against a portion of the torso immediately outwardly from the lungs of the patient whereby mucus on a wall of the lungs of the patent will be dislodged by such vibration.

21. A method of using an apparatus for massaging and compressing the lungs of a human patient, said apparatus comprising a blanket disposed substantially completely on a substantially flat surface, said blanket having an operative portion thereof comprising an entire portion that can be made to be operative, and which has at least one sealed chamber therein and adapted to have the patient lie thereon with the patient's upper torso being in juxtaposition to the sealed chamber of the blanket; and a source of compressed air operatively connected to the at least one sealed chamber for selectively inflating and deflating the blanket; the operative portion of the blanket being of a width no greater than the distance between shoulders and waist of a human person under the age of four years old; said method comprising:
(a) disposing the operative part of the blanket against an upper torso portion of a patient; and
(b) using the source of compressed air to pulsate the at least one sealed chamber to cause vibration against a portion of the torso immediately outwardly from lungs of the patient whereby mucus on a wall of the lungs of the patent will be dislodged by such vibration.

22. The method of claim 21 wherein the width of the operative portion of the blanket used to cause vibration is no more than 20 inches so that the vibration is concentrated on an area of the torso adjacent the lungs of the patient.

23. The method of claim 21 wherein the disposing step comprises:
(a) spreading out the blanket on the flat surface for massaging and compressing the lungs of the patient;
(b) laying a torso portion of a patient on the blanket; and
(c) operating the source of compressed air to cause vibration of the operative portion of the blanket.

24. The method of claim 21 wherein the disposing step comprises:
(a) laying a patient on a flat surface;
(b) spreading the operative portion of the blanket on top of the torso portion of the patient for massaging and compressing the lungs of the patient; and
(c) operating the source of compressed air to cause vibration of the operative portion of the blanket.

25. The method of claim 21 wherein the width of the operative portion of the blanket used to cause vibration is no less than 3 inches.
